(19) United States
(12) Patent Application Publication
(10) Pub. No.: US 2025/0035655 A1
(43) Pub. Date: Jan. 30, 2025

(54) SAMPLE PREPARATION VALIDATION

(71) Applicant: Beckman Coulter, Inc., Brea, CA (US)

(72) Inventors: Brahim De Jesus SAHDALA, Miami, FL (US); Rajani RAVILISETTY, Miami, FL (US); Juan Jose FERNANDEZ DE CASTRO, Miami, FL (US)

(73) Assignee: Beckman Coulter, Inc., Brea, CA (US)

(21) Appl. No.: 18/715,063

(22) PCT Filed: Dec. 8, 2022
(86) PCT No.: PCT/US2022/081168

§ 371 Date: May 30, 2024

Related U.S. Application Data
(60) Provisional application No. 63/287,327, filed on Dec. 8, 2021.
(60) Provisional application No. 63/336,815, filed on Apr. 29, 2022.

(51) Int. Cl. G01N 35/00

(52) U.S. Cl. CPC G01N 35/00663; G01N 35/00871; G01N 2035/00673; G01N 2035/00881; G01N 2035/0091

(57) ABSTRACT

A sample analysis system analyzes a sample. A panel design generates a sample preparation specification. A preparation instrument prepares a sample using the sample preparation specification to generate a prepared sample. A sample analyzer analyzes the prepared sample. A validator validates an operation of the sample analysis system. A sample analysis system has a transfer station that controls the movement of a probe with respect to a reaction plate. A probe wash module is also disclosed.

This application is being filed on Dec. 8, 2022, as a PCT International Patent application and claims the benefit of and priority to U.S. Provisional patent application Ser. No. 63/287,327, filed Dec. 8, 2021, and U.S. Provisional patent application Ser. No. 63/336,815, filed Apr. 29, 2022, the entire disclosure of which is incorporated by reference herein in its entirety.

### BACKGROUND

Before a sample is analyzed by a sample analyzer, such as a flow cytometer, the sample undergoes a preparation process to prepare the sample. For example, the sample may be stained with a labeling reagent that targets a constituent within the sample to facilitate analysis of the targeted constituent. As a more specific example, the target may be a subset of white blood cells in a blood sample, and the labeling reagent may be an antibody that attaches to the subset of white blood cells. Other sample preparation techniques can also be used. Sample analysis may be compromised if the sample is not properly prepared.

### SUMMARY

In general terms, this disclosure is directed to sample preparation. In some embodiments, and by non-limiting example, the disclosure relates to sample preparation validation. Sample preparation operation is also disclosed. Various aspects are described in this disclosure, which include, but are not limited to, the following aspects.

One aspect is a panel design system comprising a computing device and panel designer software, the panel designer software being executable by the computer to cause the panel design system to: generate a sample preparation specification; and validate the sample preparation specification.

Another aspect is a panel validation method comprising: defining a user-input specification; comparing the user-input specification with validation criteria; determining that the user-input specification does not comply with the validation criteria; generating at least one notification; and generating and outputting a sample preparation specification.

A further aspect is a sample preparation instrument comprising sample preparation hardware and sample preparation software, the sample preparation software being executable by a computer of the sample preparation hardware to cause the sample preparation instrument to: receive a sample preparation specification; define sample preparation based on at least the sample preparation specification; validate sample preparation; and generate a prepared sample based on the sample preparation.

Yet another aspect is a sample preparation method comprising: receiving a sample preparation specification; defining sample preparation based on at least the sample preparation specification; comparing the sample preparation with sample preparation validation criteria; determining that the sample preparation does not comply with the sample preparation validation criteria; generating at least one notification; and sending at least one command to sample preparation hardware.

Another aspect is a sample analysis system comprising: a panel design system that generates a sample preparation specification; a sample preparation instrument that prepares a sample using the sample preparation specification to generate a prepared sample; a sample analyzer configured to analyze the prepared sample; and at least one validator configured to validate an operation of the sample analysis system.

A further aspect is a non-transitory computer readable storage media of a sample analysis system, the computer readable storage media storing data instructions that, when executed by a processing device, cause the sample analysis system to perform operations according to any one of the methods disclosed herein.

Yet another aspect is a panel design system that compares a user-input specification with validation criteria, and generates a notification when the user-input specification does not comply with the validation criteria.

Another aspect is a sample preparation instrument that evaluates sample preparation using a sample preparation specification and generates a notification upon detecting an error.

### BRIEF DESCRIPTION OF THE DRAWINGS

FIG. 1 is a schematic block diagram depicting an example sample analysis system.

FIG. 2 is a flow chart illustrating an example method of analyzing a sample.

FIG. 3 illustrates an exemplary architecture of a computing device that can be used to implement aspects of the present disclosure.

FIG. 4 is a schematic block diagram depicting an example of a sample preparation specification system.

FIG. 5 is a flow chart illustrating an example method of validating a sample preparation specification.

FIG. 6 illustrates an example user interface for validating at least a portion of a sample preparation specification concerning white blood cells concentration.

FIG. 7 illustrates another example user interface for validating at least a portion of a sample preparation specification concerning white blood cells concentration.

FIGS. 8 is a flow chart illustrating an example method of validating at least a portion of a sample preparation specification concerning white blood cells concentration.

FIG. 9 is a flow chart illustrating an example method of validating at least a portion of a sample preparation specification concerning reagents.

FIG. 10 illustrates an example user interface for validating at least a portion of a sample preparation specification concerning reagents.

FIG. 11 illustrates another example user interface for validating at least a portion of a sample preparation specification concerning reagents.

FIG. 12 illustrates yet another example user interface for validating at least a portion of a sample preparation specification concerning reagents.

FIG. 13 illustrates a further example of a user interface for validating at least a portion of a sample preparation specification concerning reagents.

FIG. 14 is a flow chart illustrating an example method of validating at least a portion of a sample preparation specification concerning reagents.

FIG. 15 is a schematic block diagram depicting an example of sample preparation hardware.

FIG. 16 is a cross-section view of an example of sample preparation hardware.

FIG. 17 is a schematic block diagram depicting an example of a sample preparation instrument.

FIG. 18 is a flow chart illustrating an example method of validating sample preparation.

FIG. 19 illustrates an example user interface for validating sample preparation.

FIG. 20 is a flow chart illustrating an example method of validating sample preparation at sample loading procedures.

FIG. 21 is a flow chart illustrating an example method of validating sample preparation when running sample preparation procedures.

FIG. 22 is a flow chart illustrating an example method of predictively validating sample preparation.

FIG. 23 is a diagram illustrating an example configuration of the probes for using the reaction plate wells in a reaction station.

FIG. 24 is a diagram illustrating another example configuration of the probes for using the reaction plate wells in a reaction station.

FIG. 25 is a diagram depicting a portion of sample preparation hardware containing a probe wash module.

FIG. 26 is a diagram depicting a probe wash module in more detail.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

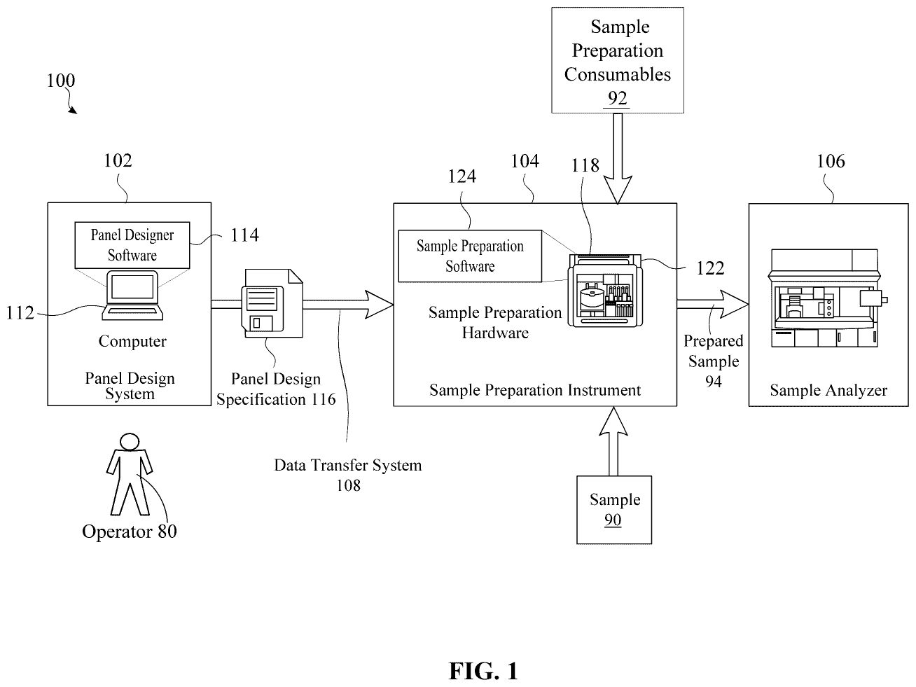

FIG. 1 is a schematic block diagram depicting an example sample analysis system 100. In the illustrated example, the sample analysis system 100 includes a panel design system 102, a sample preparation instrument 104, and a sample analyzer 106. The example panel design system 102 includes a computer 112 and panel designer software 114. The example sample preparation instrument 104 includes sample preparation hardware 122 and sample preparation software 124. Also shown in FIG. 1 is an operator 80, a sample 90, sample preparation consumables 92, a prepared sample 94, a data transfer system 108, and a sample preparation specification 116.

By way of overview, the sample analysis system 100 is operable to analyze a sample 90. Example samples can include whole blood, bone marrow, dissociated tissues, peripheral mononuclear cells, fine needle aspirates, cerebrospinal fluid, and other single cell-suspensions. Other samples or combinations of samples can be analyzed in other embodiments.

The panel design system 102 generates the sample preparation specification 116. The sample preparation instrument 104 receives the sample preparation specification 116 via a data transfer system 108. Using the sample preparation specification 116, the sample preparation instrument 104 prepares the sample 90, which sometimes involves the use of sample preparation consumables 92, and produces a prepared sample 94. The sample analyzer 106 then analyzes the prepared sample 94.

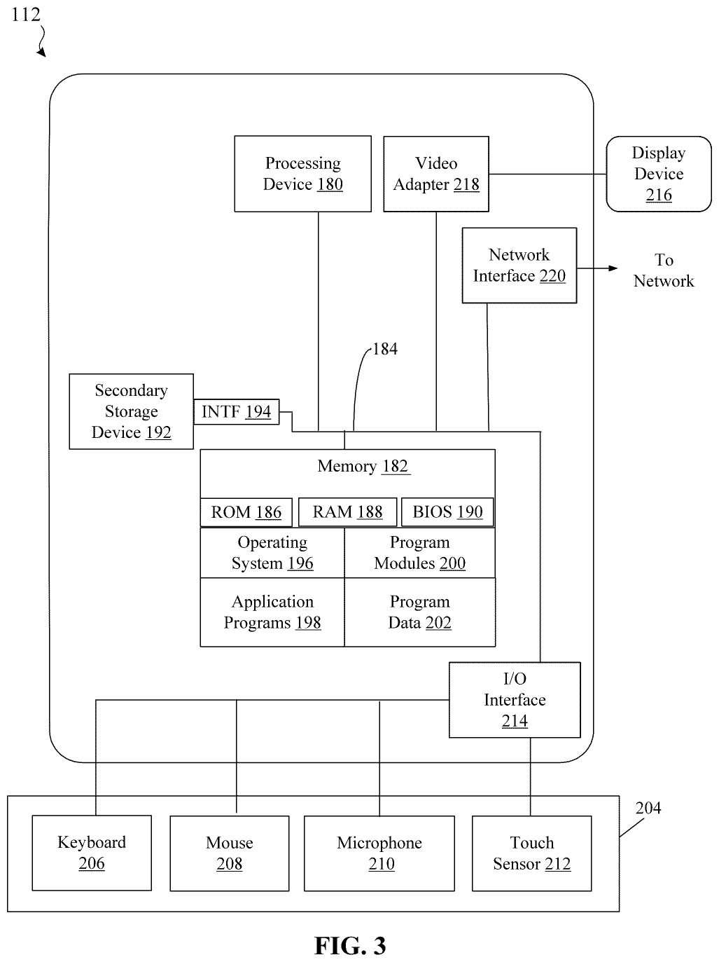

In some embodiments the panel design system 102 includes the computer 112 and the panel designer software 114, which is executed by and operates on the computer 112. An example of the computer 112 is illustrated in FIG. 3. The panel designer software 114 can receive input from an operator 80 to define the sample preparation specification 116. The sample preparation specification 116 can be subsequently used by the sample preparation instrument 104 to process the sample 90 and generate the prepared sample 94.

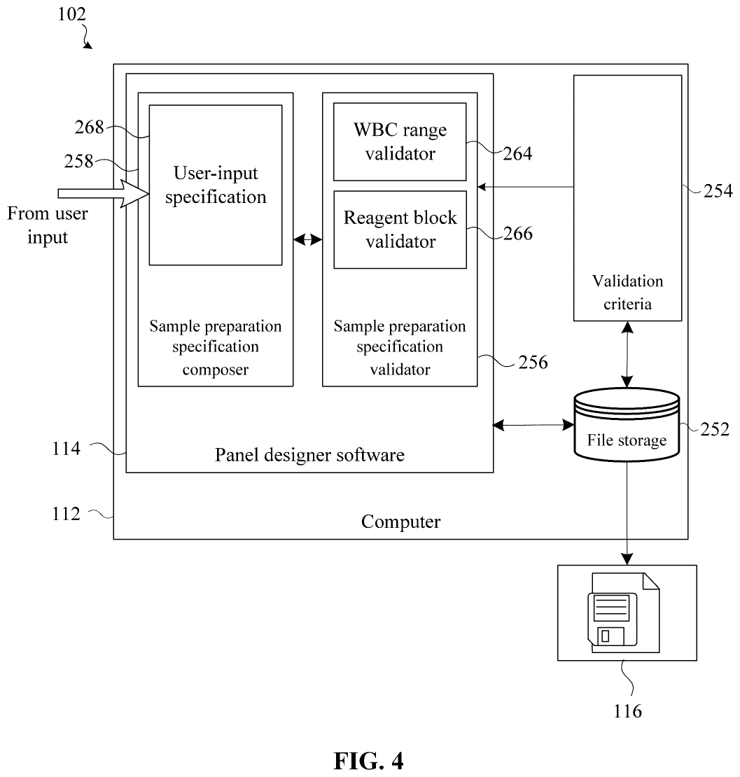

In some embodiments the panel designer software 114 also includes a sample preparation specification validator (e.g., the sample preparation specification validator 256, shown in FIG. 4), which operates to validate the sample preparation specification 116. The sample preparation specification 116 can then be provided to the sample preparation instrument 104.

Examples of the panel design system 102 are illustrated and described in further detail herein with reference to FIGS. 4-14.

The data transfer system 108 can be used to transfer the sample preparation specification 116 from the panel design system 102 to the sample preparation instrument 104. In one example the data transfer system 108 is a data communication network such as the Internet or other network or combination of networks. In another example, the data transfer system 108 can be a manual transfer method. For example, the sample preparation specification 116 can be saved on a computer-readable storage device, such as a disc or flash drive, and then delivered to the sample preparation instrument 104, such as by the operator 80.

In the illustrated example, the sample preparation instrument 104 includes sample preparation hardware 122 and sample preparation software 124. In some embodiments the sample preparation software 124 is executed by and operates on the sample preparation hardware 122 (such as using a sample preparation computer 118 of the sample preparation hardware 122).

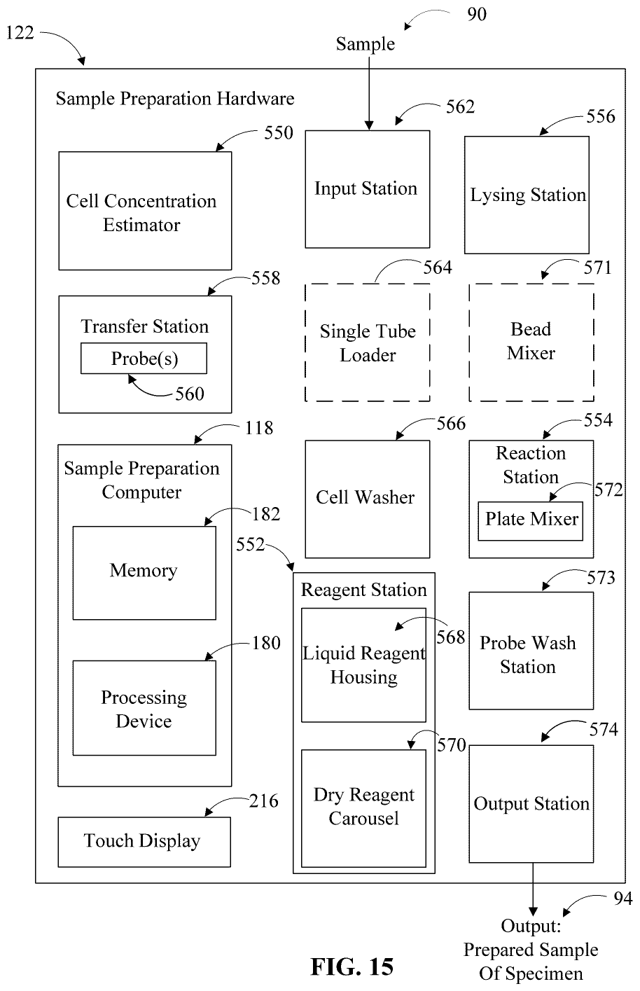

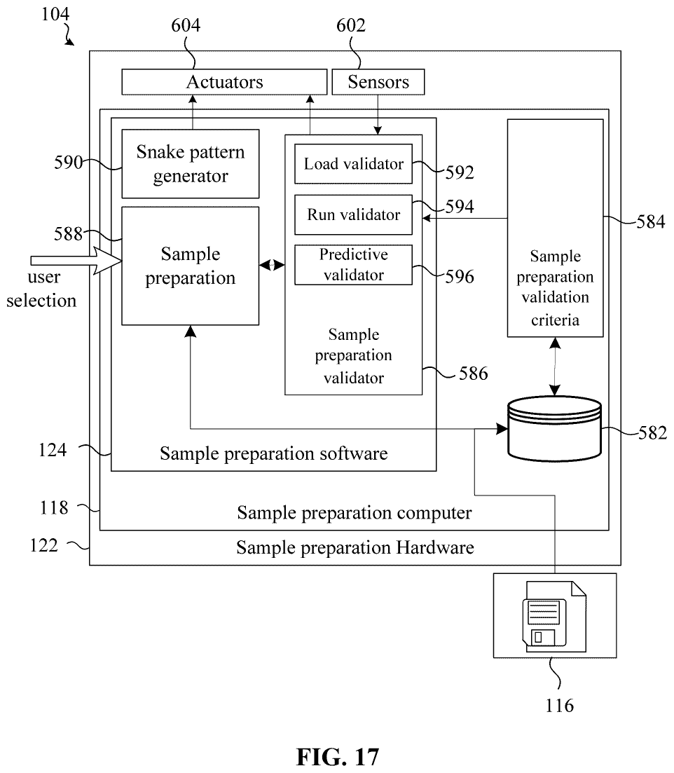

In some embodiments, the sample preparation software 124 includes a sample preparation validator (e.g., the sample preparation validator 586, shown in FIG. 17), which operates to validate the sample preparation. For example, some sample preparations utilize sample preparation consumables 92, and so the sample preparation validator can operate to check whether the required sample preparation consumables 92 are present and available in sufficient quantities. The sample preparation software 124 is operable to communicate with the sample preparation hardware 122, and to cause the sample preparation hardware 122 to prepare the sample 90 with the sample preparation consumables 92 according to the validated sample preparation. In another embodiment, the sample preparation software 124 is operable to validate the status of the sample preparation hardware 122 with regards to the sample preparation specification 116. Examples of the sample preparation instrument 104 are illustrated and described in further detail herein with reference to FIGS. 15-26.

The sample analyzer 106 is an instrument that is operable to analyze the prepared sample 94. In some embodiments the sample analyzer 106 is a laboratory instrument. One specific example of a sample analyzer 106 is a flow cytometer. Another example of a sample analyzer is a hematology analyzer. Other embodiments include other types of sample analyzers.

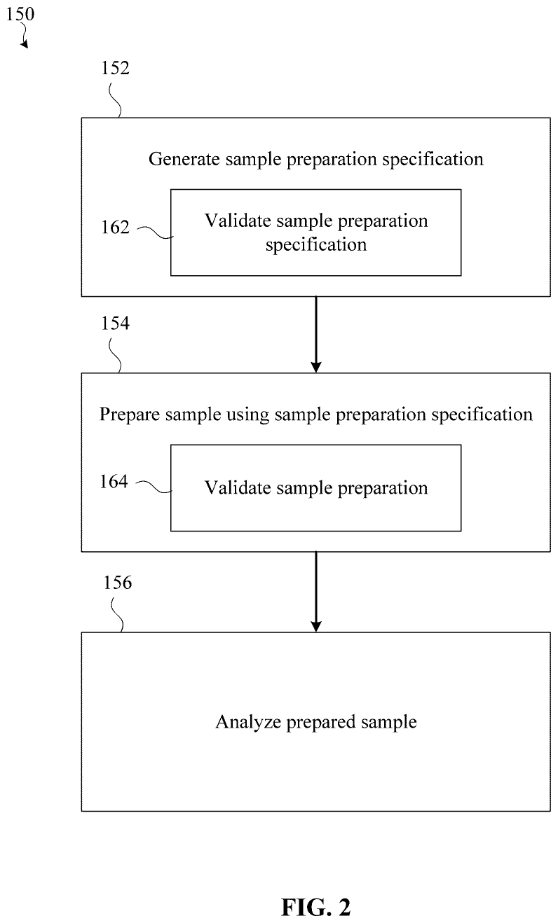

FIG. 2 is a flow chart illustrating an example method 150 of analyzing a sample. In one example, the method 150 is operated by the sample analysis system 100, shown in FIG. 1. In this example, the method 150 includes operations 152, 154, and 156. Some embodiments also include one or more of operations 162 and 164.

The operation 152 is performed to generate a sample preparation specification 116. In some embodiments the operation 152 is performed by the panel design system 102, such as shown in FIG. 1.

The operation 154 is performed to prepare a sample 90 using the sample preparation specification 116 (shown in FIG. 1). In some embodiments the operation 154 is performed by the sample preparation instrument 104, such as shown in FIG. 1.

The operation 156 is performed to analyze the prepared sample 94 that was prepared by the operation 154. In some embodiments the operation 156 is performed by the sample analyzer 106, such as shown in FIG. 1.

Further, some embodiments include one or more validation operations. For example, operation 152 can also include an operation 162. The operation 162 is performed to validate the sample preparation specification 116.

As another example, the operation 154 can include an operation 164. The operation 164 is performed to validate the sample preparation.

FIG. 3 illustrates an exemplary architecture of a computing device that can be used to implement aspects of the present disclosure, including any of the computer 112 (FIGS. 1 and 4) and the sample preparation computer 118 of the sample preparation hardware 122 (FIGS. 1, 15 and 17). The computing device illustrated in FIG. 3 can be used to execute the operating system, application programs, and software modules (including the software engines) described herein. By way of example, the computing device will be described below as the computer 112. To avoid undue repetition, this description of the computing device will not be separately repeated herein for each of the other computing devices, but such devices can also be configured as illustrated and described with reference to FIG. 3.

The computer 112 includes, in some embodiments, at least one processing device 180, such as a central processing unit (CPU). A variety of processing devices are available from a variety of manufacturers, for example, Intel or Advanced Micro Devices. In this example, the computer 112 also includes a system memory 182, and a system bus 184 that couples various system components including the system memory 182 to the processing device 180. The system bus 184 is one of any number of types of bus structures including a memory bus, or memory controller; a peripheral bus; and a local bus using any of a variety of bus architectures.

Examples of computing devices suitable for the computer 112 include a server computer, a desktop computer, a laptop computer, a tablet computer, a mobile computing device (such as a smart phone, an iPod® or iPad® mobile digital device, or other mobile devices), or other devices configured to process digital instructions.

The system memory 182 includes read only memory 186 and random access memory 188. A basic input/output system 190 containing the basic routines that act to transfer information within the computer 112, such as during start up, is typically stored in the read only memory 186.

The computer 112 also includes a secondary storage device 192 in some embodiments, such as a hard disk drive, for storing digital data. The secondary storage device 192 is connected to the system bus 184 by a secondary storage interface 194. The secondary storage devices 192 and their associated computer readable media provide nonvolatile storage of computer readable instructions (including application programs and program modules), data structures, and other data for the computer 112.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. Additionally, such computer readable storage media can include local storage or cloud-based storage.

A number of program modules can be stored in secondary storage device 192 or memory 182, including an operating system 196, one or more application programs 198, other program modules 200 (such as the software engines described herein), and program data 202. One example of the application programs 198 is the sample preparation software 124. The computer 112 can utilize any suitable operating system, such as Microsoft Windows™, Google Chrome™, Apple OS, and any other operating system suitable for a computing device.

In some embodiments, a user provides inputs to the computer 112 through one or more input devices 204. Examples of input devices 204 include a keyboard 206, mouse 208, microphone 210, and touch sensor 212 (such as a touchpad or touch sensitive display). Other embodiments include other input devices 204. The input devices are often connected to the processing device 180 through an input/output interface 214 that is coupled to the system bus 184. These input devices 204 can be connected by any number of input/output interfaces, such as a parallel port, serial port, game port, or a universal serial bus. Wireless communication between input devices and the interface 214 is possible as well, and includes infrared, BLUETOOTH® wireless technology, 802.11a/b/g/n, cellular, or other radio frequency communication systems in some possible embodiments.

In this example embodiment, a display device 216, such as a monitor, liquid crystal display device, projector, or touch sensitive display device, is also connected to the system bus 184 via an interface, such as a video adapter 218. In addition to the display device 216, the computer 112 can include various other peripheral devices (not shown), such as speakers or a printer.

When used in a local area networking environment or a wide area networking environment (such as the Internet), the computer 112 is typically connected to the network through a network interface 220, such as an Ethernet interface. Other possible embodiments use other communication devices. For example, some embodiments of the computer 112 include a modem for communicating across the network.

The computer 112 typically includes at least some form of computer readable media. Computer readable media includes any available media that can be accessed by the computer 112. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computer 112. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 3 is also an example of programmable electronics, which may include one or more such computing devices, and when multiple computing devices are included, such computing devices can be coupled together with a suitable data communication network so as to collectively perform the various functions, methods, or operations disclosed herein.

FIG. 4 is a schematic block diagram illustrating an example of the panel design system 102, which is illustrated and described in FIG. 1. In this example, the panel design system 102 includes the computer 112. In some embodiments, the computer 112 includes the panel designer software 114, a file storage 252, and validation criteria 254. The panel designer software 114 is executed by and operates on the computer 112. Also shown in this illustrated example is the sample preparation specification 116. In some embodiments, the panel designer software 114 includes a sample preparation specification validator 256 and a sample preparation specification composer 258. The example sample preparation specification validator 256 includes a WBC range validator 264 and a reagent block validator 266. The example sample preparation specification composer 258 includes a user-input specification 268.

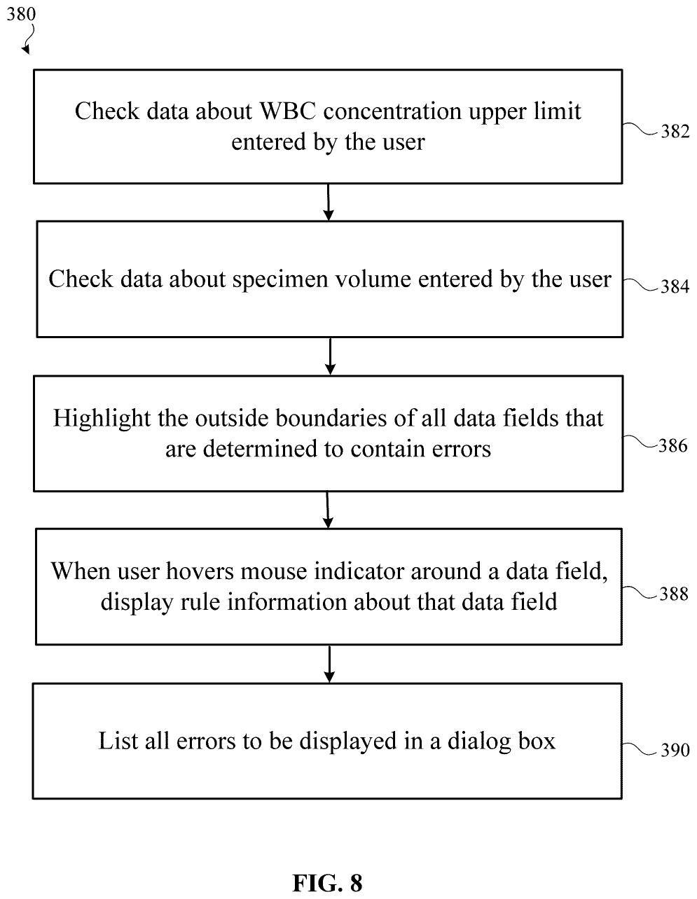

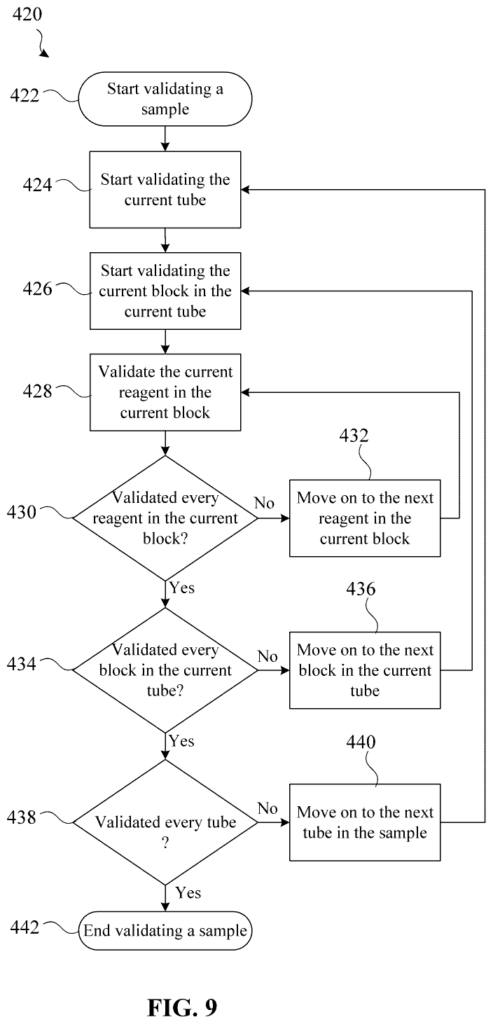

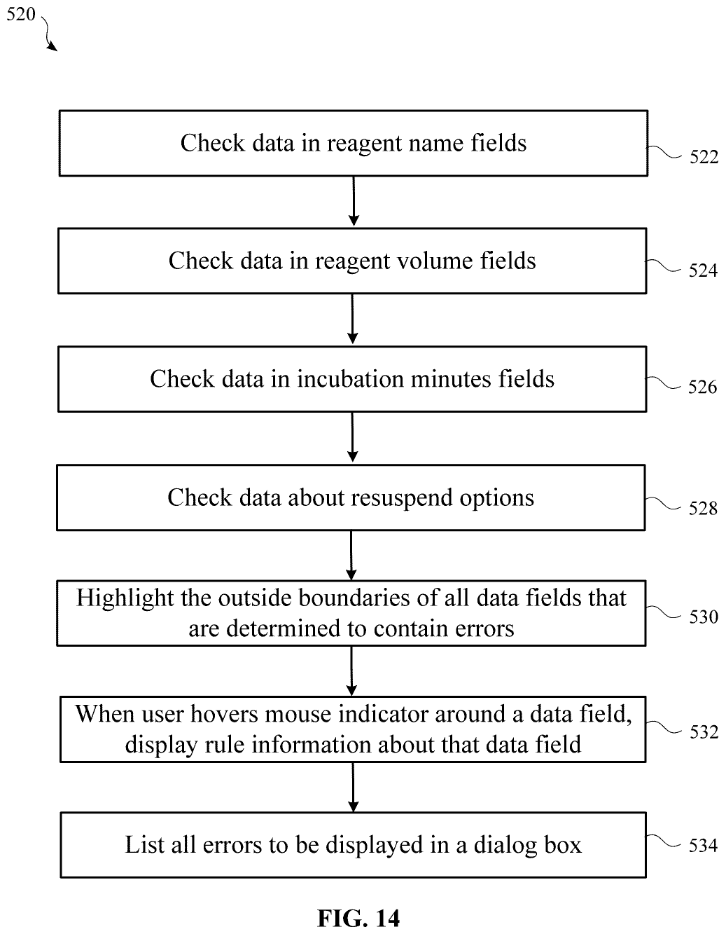

In some embodiments, the user-input specification 268 can, for example, be from a user's manual input. The sample preparation specification validator 256 then operates to validate the user-input specification 268 against the validation criteria 254 using the WBC range validator 264 and the reagent block validator 266, respectively. In this illustrated example, the validation criteria 254 is provided by the file storage 252. After validation, the panel designer software 114 is operable to generate the sample preparation specification 116, and to output the sample preparation specification 116 via the file storage 252. An example method of using the WBC range validator 264 is illustrated and described in further detail herein with reference to FIG. 8. Other example methods of using the reagent block validator 266 is illustrated and described in further detail herein with reference to FIG. 9 and FIG. 14.

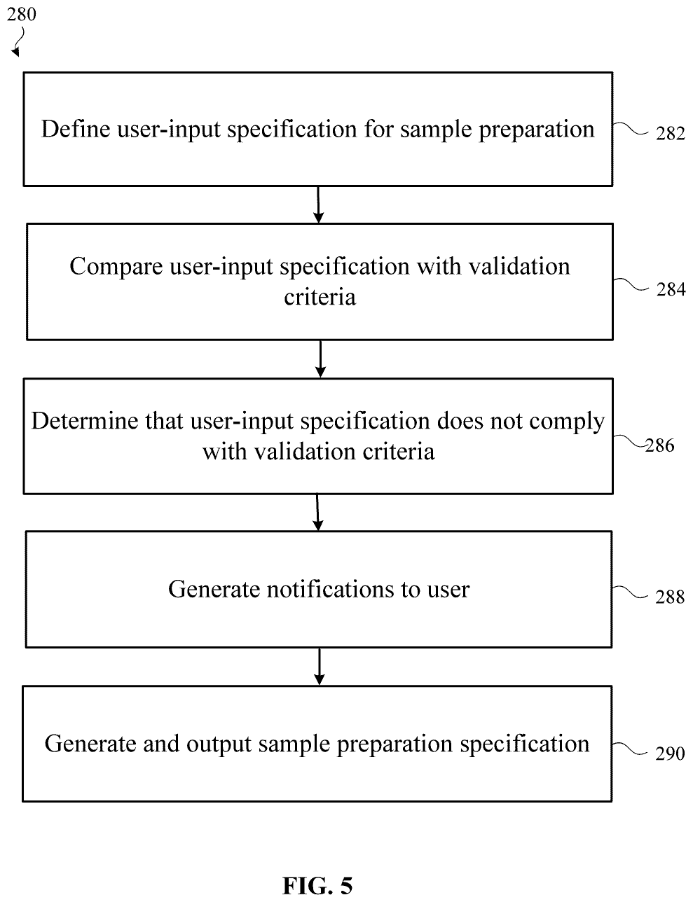

FIG. 5 is a flow chart illustrating an example method 280 of validating a sample preparation specification. In one example, the method 280 is operated by the panel design system 102, shown in FIG. 4. In this example, the method 280 includes operations 282, 284, 286, 288, and 290.

The operation 282 is performed to define the user-input specification 268 for sample preparation. In some embodiments the operation 282 is performed by the sample preparation specification composer 258, such as shown in FIG. 4.

The operation 284 is performed to compare the user-input specification 268 with the validation criteria 254. In some embodiments the operation 284 is performed by the sample preparation specification validator 256, such as shown in FIG. 4.

The operation 286 is performed to determine that the user-input specification 268 does not comply with the validation criteria 254. In some embodiments the operation 286 is performed by the sample preparation specification validator 256, such as shown in FIG. 4.

The operation 288 is performed to generate notifications to alert user of errors found by validating the user-input specification 268, according to the determination made by the operation 286. In some embodiments the operation 288 is performed by the sample preparation specification validator 256, such as shown in FIG. 4.

The operation 290 is performed to generate and output the sample preparation specification 116. In some embodiments the operation 290 is performed by the sample preparation specification validator 256, such as shown in FIG. 4.

In some further embodiments, the sample preparation specification 116 generated and output by the operation 290 is configured to contain an indicator. If any portion of the user-input specification 268 does not comply with the validation criteria 254, the indicator operates to indicate an error status for the definition of the user-input specification 268. In some examples, such an indicator can be a flag variable having a value that may be “true” or “false.” To indicate an error status, the flag variable's value will be “true”; otherwise, the flag variable's value will be “false.” In other examples, the sample preparation specification 116 contains a hash code. In some embodiments, the hash code is generated by processing the sample preparation specification 116. The hash code can be used to verify that no unauthorized modifications have been made to the sample preparation specification 116. For instance, it can only be edited by using the panel designer software 114.

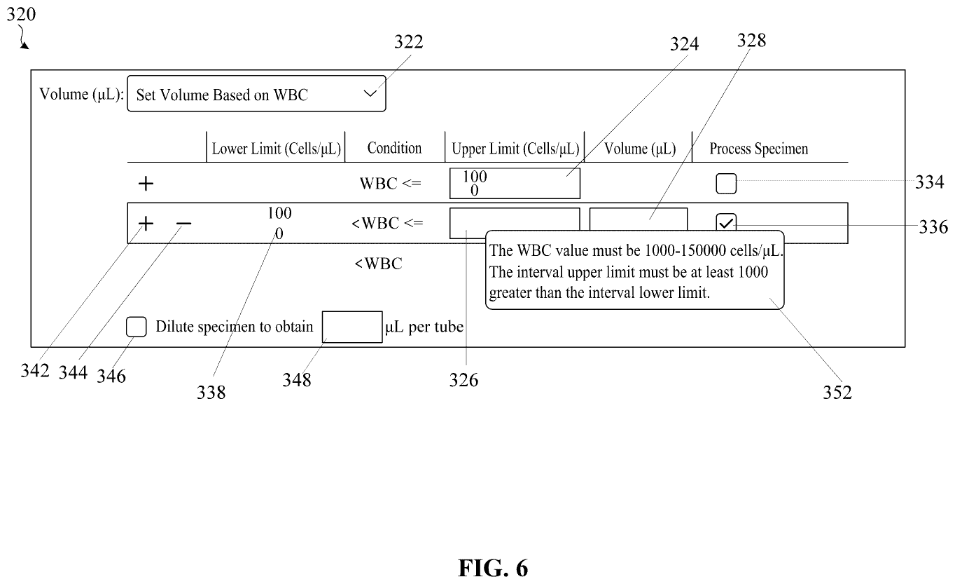

FIG. 6 illustrates and describes an example user interface 320 for using the WBC range validator 264, such as shown in FIG. 4, to check and validate at least a portion of the user-input specification 268 in the sample preparation specification composer 258, the portion concerning white blood cells. In this illustrated example, the user interface 320 displays a drop-down menu selector 322. With the drop-down menu selector 322, a user can select to expand design options for the user to input the portion of the user-input specification 268 concerning white blood cells. In some embodiments, the design options include a first WBC upper limit field 324 and a second WBC upper limit field 326. After the user, for example, inputs data into the fields 324 and 326, the WBC range validator 264 operates to check the data against some predetermined rules. In some examples, the rules checked by the WBC range validator 264 include that the data in the fields 324 and 326 must be within a specified range (for instance, within 1000-150000 cells per micro liter). In some further examples, the rules include that the data in the second WBC upper limit field 326 must be at least 1000 cells per micro liter greater than the data in the first WBC upper limit field 324.

In some further embodiments, the example user interface 320 includes a volume field 328 corresponding to the second WBC upper limit field 326, a first process checkbox 334 corresponding to the first WBC upper limit field 324, and a second process checkbox 336 corresponding to the second WBC upper limit field 326. For both checkboxes 334 and 336, the user has the option to check or not to check. The user's checking the first process checkbox 334 indicates that the user specifies to process a specimen if the white blood cells concentration found in the specimen is lower or equal to the data in the first WBC upper limit field 324. The user's checking the second process checkbox 336 indicates that the user specifies to process a specimen if the white blood cells concentration found in the specimen is lower or equal to the data in the second WBC upper limit field 326, but higher than the data in the first WBC upper limit field 324. Some other examples of the rules checked by the WBC range validator 264 include that, if one of the process checkboxes is checked by the user (for example, the second checkbox 336), the data in the corresponding volume field 328 must be within a specified range (for instance, within 25-400 micro liters).

In some other embodiments, the example user interface 320 includes an add row button 342 and a delete row button 344. The user has the option to delete a row of data by clicking the delete row button 344 and add a row of data by clicking the add row button 342. Some other examples of the rules checked by the WBC range validator 264 include that, if additional rows of data are added by the user, the specimen volume is less than the specimen volume from the row before.

In some further embodiments, the example user interface 320 includes a dilute checkbox 346 and a dilution volume field 348. The user has the option to input data in the dilution volume field 348 only if the user has checked the dilute checkbox 346.

In some other embodiments, the WBC range validator 264 operates to cause the outside boundary of any field having data that is checked by the WBC range validator 264 and determined to be erroneous to be highlighted (for instance, using a color-coded indication, such as to change color to red). This operation of the WBC range validator 264 is configured to notify the user of the fields in which the user has input erroneous data.

In some further embodiments, the WBC range validator 264 operates to cause the example user interface 320 to display an information box 352 containing rules information about the data to be input in a field, if and when the user hovers the mouse indicator of the mouse 208, such as shown in FIG. 3, in an area close to the corresponding data field. A mouse pointer is an example of a pointer, and other pointers can also be used in other embodiments (e.g., touch input pointers, track pad pointers, and the like).

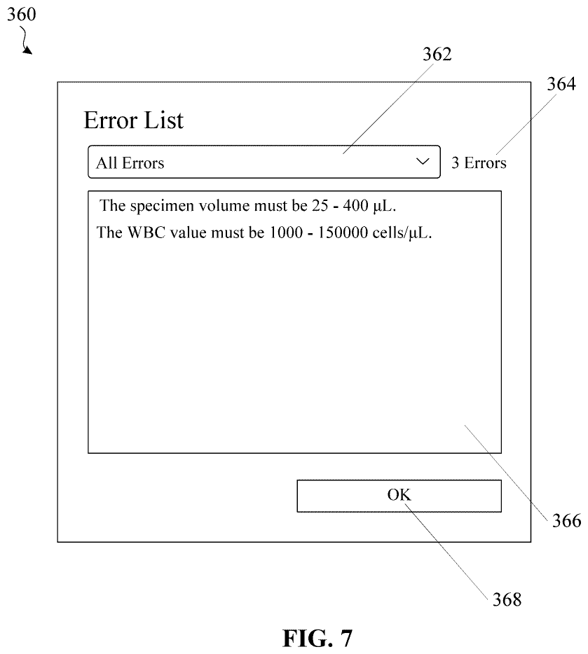

FIG. 7 illustrates and describes an example dialog box 360 for using the WBC range validator 264, such as shown in FIG. 4, to display all errors found by the WBC range validator 264 when validating the portion of the user-input specification 268 concerning white blood cells. In this illustrated example, the dialog box 360 displays a drop-down list selector 362 and an error count indicator 364. The error count indicator 364 operates to notify the user the number of errors that the WBC range validator 264 has found after validating the portion of the user-input specification 268 concerning white blood cells.

In some embodiments, with the drop-down list selector 362, the user can select to cause the example dialog box 360 to display a WBC error list 366 containing the detailed information about all errors that the WBC range validator 264 has found after validating the portion of the user-input specification 268 concerning white blood cells. In other examples, the dialog box 360 also displays a WBC error list dismiss button 368. By clicking the WBC error list dismiss button 368, the user can select to cause the example dialog box 360 to close out.

FIG. 8 is a flow chart illustrating an example method 380 of validating a portion of a sample preparation specification concerning white blood cells. In one example, the method 380 is operated by the WBC range validator 264, shown in FIG. 4. In this example, the method 380 includes operations 382, 384, 386, 388, and 390.

In this illustrated example, the operation 382 is performed to check data about WBC concentration upper limit entered by the user. The operation 384 is performed to check data about specimen volume entered by the user. To highlight the errors found by the operations 382 and 384, the operation 386 is performed to highlight the outside boundaries (for instance, change color to red) of all data fields that are determined to contain errors. To provide more relevant information to the user, the operation 388 is performed to display rule information about the data field, around which the user hovers the user's mouse indicator. To summarize all errors found in a notification, the operation 390 is performed to list all errors to be displayed in a dialog box.

FIG. 9 is a flow chart illustrating an example method 420 of validating at least a portion of a sample preparation specification concerning reagents. Examples of reagents include antibody, lyse, wash buffer, diluent, etc. In one example, the method 420 is operated by the reagent block validator 266, shown in FIG. 4. In some embodiments, the reagent block validator 266 operates to validate at least a portion of the user-input specification 268 concerning reagents, the portion being organized by the panel designer software 114 to have multiple hierarchies. The user-input specification 268 for a sample includes the specification for at least one tube. Each tube's specification includes the specification for at least one block, each of the block's specification further including the specification for at least one reagent.

In the illustrated example, the method 420 includes operations 422, 424, 426, 428, 430, 432, 434, 436, 438, 440, and 442. The operation 422 is performed to start validating the portion of a sample preparation specification concerning reagents. Next, the operation 424 is performed to start validating the specification for a current tube as part of the current sample. Then, the operation 426 is performed to start validating the specification for a current block as part of the current tube. Following the operation 426, the operation 428 is performed to validate the specification for a current reagent as part of the current block. After the operation 428, the operation 430 is performed to check whether the validation of every reagent in the current block has been completed. If no, the operation 432 is performed to move on to the next reagent in the current block and prepare to validate that reagent as the new current reagent. Subsequently, the operation 428 is re-performed to validate the specification for the new current reagent.

If the answer to the operation 430 is yes, the operation 434 is performed to check whether the validation of every block in the current block has been completed. If no, the operation 436 is performed to move on to the next block in the current tube and prepare to validate that block as the new current block. Subsequently, the operation 426 is re-performed to start validating the specification for the new current block.

If the answer to the operation 434 is yes, the operation 438 is performed to check whether the validation of every tube in the current sample has been completed. If no, the operation 440 is performed to move on to the next tube in the current sample and prepare to validate that tube as the new current tube. Subsequently, the operation 424 is re-performed to start validating the specification for the new current tube.

If the answer to the operation 438 is yes, the operation 442 is performed to end validating the current sample and mark its completion.

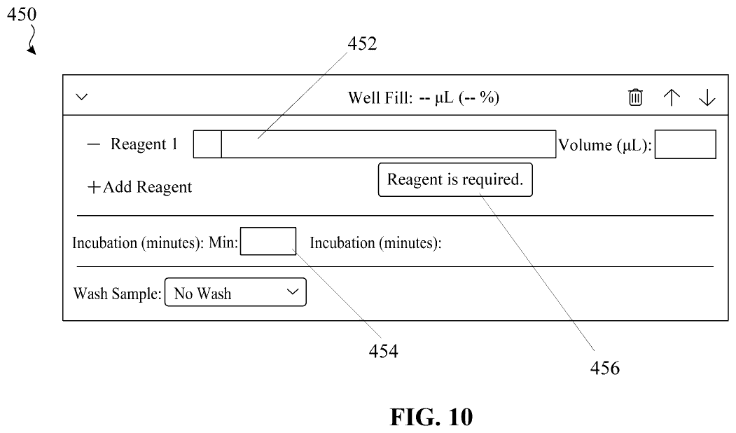

FIG. 10 illustrates and describes an example user interface 450 for using the reagent block validator 266, such as shown in FIG. 4, to check and validate at least a portion of the user-input specification 268, the portion concerning reagents. In this illustrated example, the user interface 450 includes a reagent name field 452. In some examples, the rules checked by the reagent block validator 266 include that the reagent name field 452 requires an input of a regent name or an identification string, etc. In some embodiments, the user interface 450 includes a minimum incubation minutes field 454.

In some other embodiments, the reagent block validator 266 operates to cause the outside boundary of any field having data that is checked by the reagent block validator 266 and determined to be erroneous to be highlighted (for instance, change color to red). This operation of the reagent block validator 266 is configured to notify the user of the fields in which the user has input erroneous data.

In some further embodiments, the reagent block validator 266 operates to cause the example user interface 450 to display an information box 456 containing rules information about the data to be input in a field, if and when the user hovers the mouse indicator of the mouse 208, such as shown in FIG. 3, in an area close to the corresponding data field.

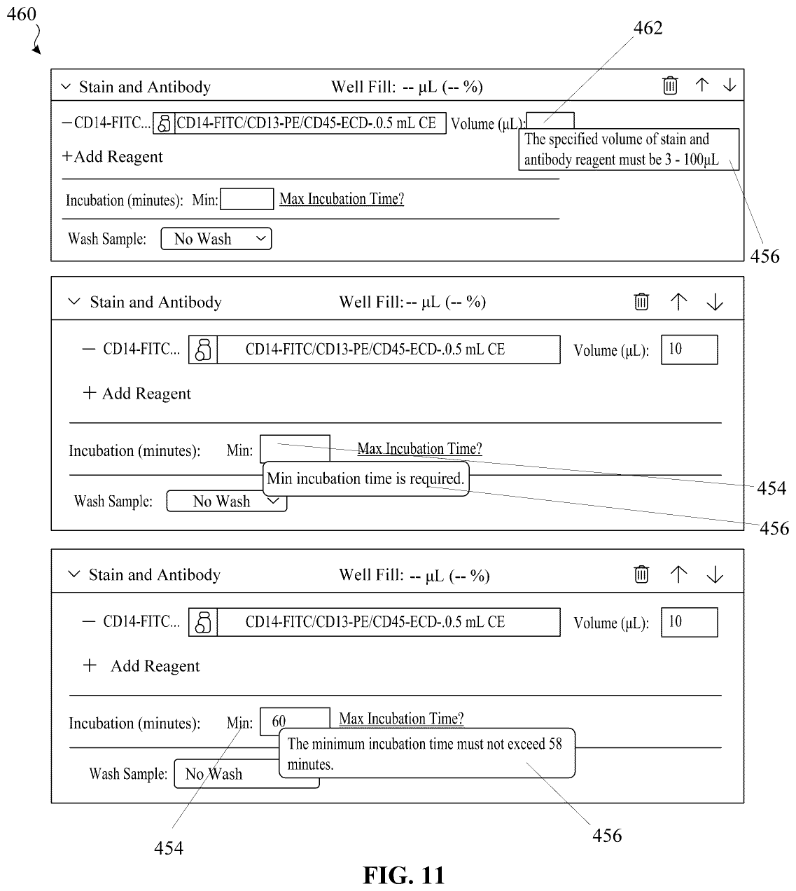

FIG. 11 illustrates and describes another example user interface 460 for using the reagent block validator 266, such as shown in FIG. 4, to check and validate at least a portion of the user-input specification 268, the portion concerning reagents. In this illustrated example, the user interface 460 includes a reagent volume field 462. In some examples, the rules checked by the reagent block validator 266 include that the data in the reagent volume field 462 must be within a specified range (for instance, between 3-100 micro liters).

In some embodiments, the user interface 460 includes the minimum incubation minutes field 454. In some examples, the rules checked by the reagent block validator 266 include that the minimum incubation minutes field 454 requires an input of a number, for instance. Another example of the rules checked by the reagent block validator 266 is that the data in the minimum incubation minutes field 454 must not exceed 58, when the units for the data are minutes.

In some other embodiments, the reagent block validator 266 operates to cause the outside boundary of any field having data that is checked by the reagent block validator 266 and determined to be erroneous to be highlighted (for instance, change color to red). This operation of the reagent block validator 266 is configured to notify the user of the fields in which the user has input erroneous data.

In further embodiments, the reagent block validator 266 operates to cause the example user interface 460 to display the information box 456 containing rules information about the data to be input in a field, if and when the user hovers the mouse indicator of the mouse 208, such as shown in FIG. 3, in an area close to the corresponding data field.

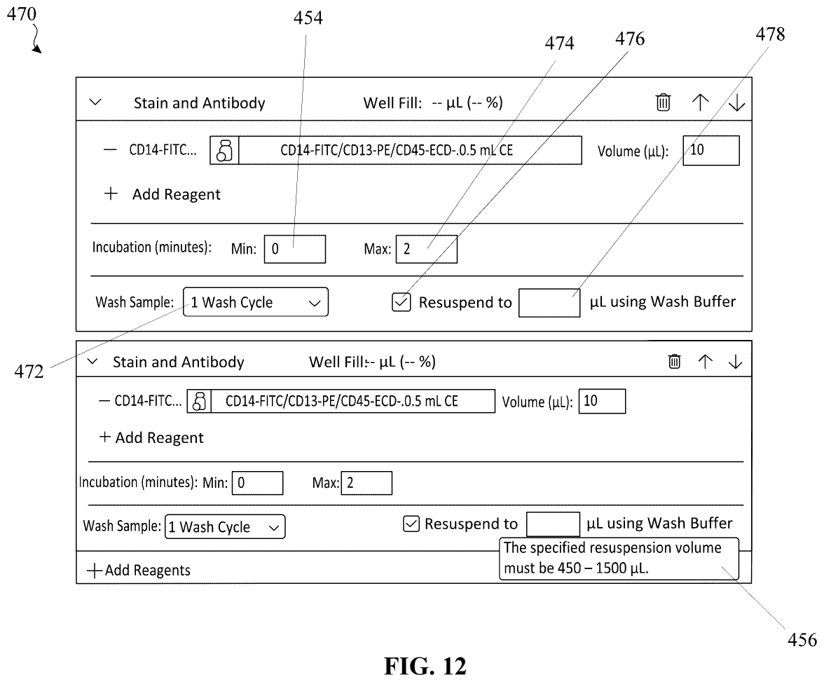

FIG. 12 illustrates and describes yet another example user interface 470 for using the reagent block validator 266, such as shown in FIG. 4, to check and validate at least a portion of the user-input specification 268, the portion concerning reagents. In this illustrated example, the user interface 470 includes a wash sample selector 472. With the wash sample selector 472, the user can select to cause the user-input specification 268 to include and record the user's choice among various wash cycle options. Some examples of the wash cycle options include “no wash” or “1 wash cycle”, etc. If the user selects an option that is not “no wash”, the user interface 470 can display a maximum incubation minutes field 474 near the minimum incubation minutes field 454.

In further embodiments, if the user selects an option for the wash sample selector 472 that is not “no wash”, the user interface 470 can display a wash resuspend checkbox 476. For the wash resuspend checkbox 476, the user has the option to check or not to check. The user's checking the wash resuspend checkbox 476 indicates that the user specifies that the sample will be resuspended using, for instance, wash buffer. If the wash resuspend checkbox 476 is checked by the user, the user interface 470 can display a resuspend volume field 478. Some examples of the rules checked by the reagent block validator 266 include that the data in the resuspend volume field 478 must be within a specified range (for instance, between 450-1500 micro liters).

In some other embodiments, the reagent block validator 266 operates to cause the outside boundary of any field having data that is checked by the reagent block validator 266 and determined to be erroneous to be highlighted (for instance, change color to red). This operation of the reagent block validator 266 is configured to notify the user of the fields in which the user has input erroneous data.

In yet further embodiments, the reagent block validator 266 operates to cause the example user interface 470 to display the information box 456 containing rules information about the data to be input in a field, if and when the user hovers the mouse indicator of the mouse 208, such as shown in FIG. 3, in an area close to the corresponding data field.

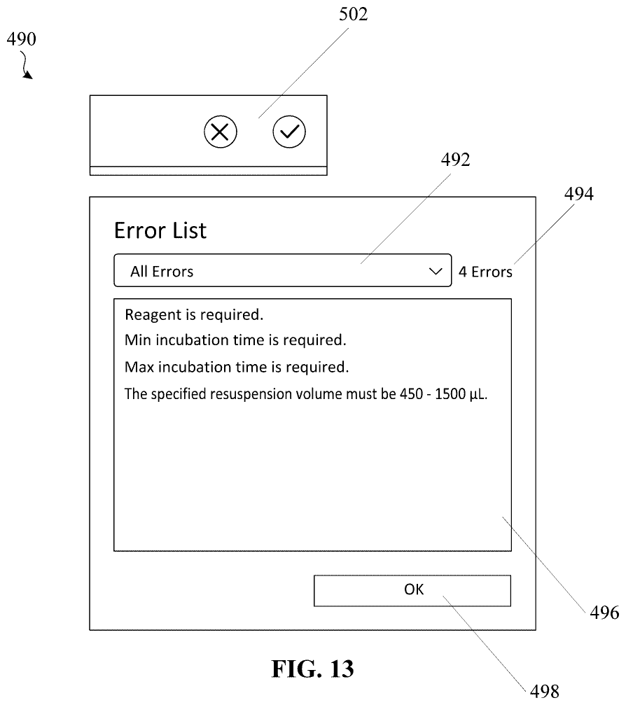

FIG. 13 illustrates and describes another example dialog box 490 for using the reagent block validator 266, such as shown in FIG. 4, to display all errors found by the reagent block validator 266 when validating the portion of the user-input specification 268 concerning reagents. In this illustrated example, the dialog box 490 displays a reagent drop-down list selector 492 and a reagent error count indicator 494. The reagent error count indicator 494 operates to notify the user the number of errors that the reagent block validator 266 has found after validating the portion of the user-input specification 268 concerning reagents.

In some embodiments, the example dialog box 490 includes an error status indicator 502. If there is at least one error found by the reagent block validator 266, the error status indicator 502 operates to indicate an error status by, for instance, displaying an icon.

In further embodiments, with the reagent drop-down list selector 492, the user can select to cause the example dialog box 490 to display a reagent error list 496 containing the detailed information about all errors that the reagent block validator 266 has found after validating the portion of the user-input specification 268 concerning reagents. In other examples, the dialog box 490 also displays a reagent error list dismiss button 498. By clicking the reagent error list dismiss button 498, the user can select to cause the example dialog box 490 to close out.

FIG. 14 is a flow chart illustrating an example method 520 of validating a portion of a sample preparation specification concerning reagents. In one example, the method 520 is operated by the reagent block validator 266, shown in FIG. 4. In this example, the method 520 includes operations 522, 524, 526, 528, 530, 532, and 534.

In this illustrated example, the operation 522 is performed to check the data in reagent name fields. The operation 524 is performed to check the data in reagent volume fields. The operation 526 is performed to check the data in incubation minutes fields. The operation 528 is performed to check the data about resuspend options.

In some embodiments, to highlight the errors found by the operations 522, 524, 526, and 528, the operation 530 is performed to highlight the outside boundaries (for instance, change color to red) of all data fields that are determined to contain errors. To provide more relevant information to the user, the operation 532 is performed to display rule information about the data field, around which the user hovers the user's mouse indicator. To summarize all errors found in a notification, the operation 534 is performed to list all errors to be displayed in a dialog box.

FIG. 15 is a schematic block diagram illustrating and describing an example of the sample preparation hardware 122 (also described in FIG. 1). In this illustrated example, the sample preparation hardware 122 includes a cell concentration estimator 550, a reagent station 552, a reaction station 554, and a lysing station 556. In some embodiments, the sample preparation hardware 122 can further include a transfer station 558 comprising one or more probes 560, the sample preparation computer 118 comprising a memory 182 and at least one processing device 180, a touch display 216, an input station 562, a single tube loader 564, a cell washer 566, a liquid reagent housing 568 and a dry reagent carousel 570 within the reagent station 552, a bead mixer 571, a plate mixer 572 within the reaction station 554, a probe wash station 573, and an output station 574.

The probes 560 of the transfer station 558 can aspirate, transport, and dispense various substances among components of the sample preparation instrument 104. The substances can include specimens, labeling reagents, lytic reagents, diluent reagents, and buffers, among other examples. The probes 560 can pierce capped or sealed tubes, vials, cartridges, bottles, or other similar containers to aspirate the substances within or can be inserted into open-top tubes, vials, cartridges, bottles, or other similar open-top containers.

The sample preparation computer 118, described in greater detail in FIG. 3, is a controller of the sample preparation instrument 104, as well as an information processor. In some examples, the sample preparation computer 118 can store previously authored panels received from a remote computing device. For example, a user, such as a laboratory technician or analyst, can utilize a panel authoring and management application executing on the remote computing device to author panels for different types of specimens and different types of analysis for execution by the sample preparation instrument 104. The authored panels can then be transmitted to the sample preparation instrument 104 over a network or wired connection or a physical memory device such as a USB drive. In other examples, the sample preparation computer 118 can execute a panel authoring and management application to allow the user to author the panel using the sample preparation instrument 104 (e.g., via user interfaces provided via the touch display 216), where the authored panels are subsequently stored by the sample preparation computer 118. Each panel can define a particular workflow to be performed when preparing a sample of a specimen for the panel. For example, the panel can include a set of rules defining the particular workflow associated with sample preparation, hereinafter referred to as the set of predefined rules.

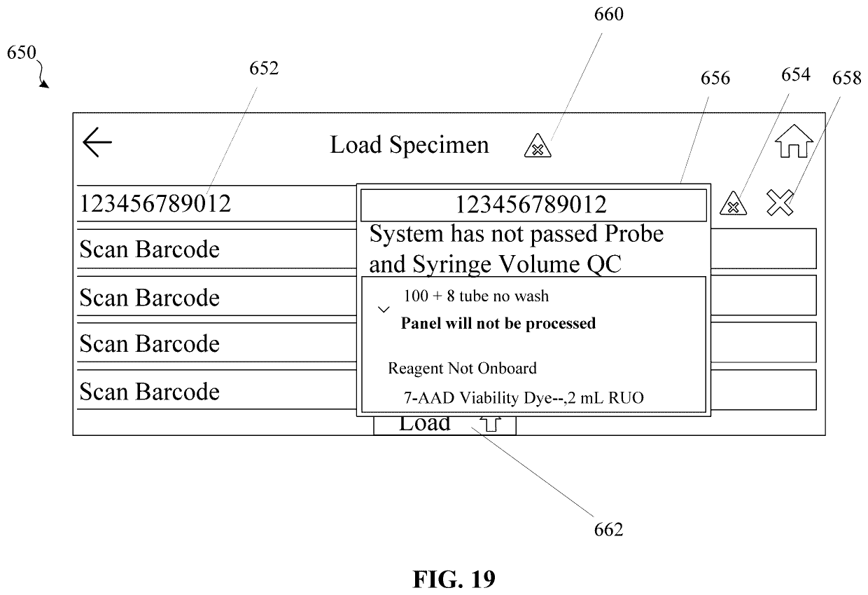

The sample preparation computer 118, by operating the sample preparation software 124, can provide the authored panels for display through a specimen loading user interface shown and described with reference to FIG. 19 to enable a user to select one or more of the panels to pair with a received specimen. In some examples, the specimen loading user interface can be provided through the touch display 216 of the sample preparation instrument 104. The selected one or more panels can then be used to manage the operations of the sample preparation instrument 104 while preparing the sample. For example, the set of predefined rules associated with each panel can provide an order of operations, a type and a volume amount of specimen and reagent involved in each operation, a time associated with each operation, and components of the sample preparation instrument 104 involved in each operation, among other examples. Based on the selected one or more panels, the sample preparation computer 118 can generate signals for transmission to respective components of the sample preparation instrument 104 that provide instructions for executing the operations.

Additionally, the sample preparation computer 118 can operate in conjunction with the cell concentration estimator 550 to determine the white blood cell concentration in the specimen. Based on the set of predefined rules associated with the one or more selected panels, the sample preparation computer 118 can further determine whether or not to process the specimen, a sample volume of the specimen based on the white blood cell concentration estimate, and whether the specimen is to be diluted, among other determinations discussed herein.

In some examples, the input station 562 receives an input cassette that has been inserted into the sample preparation instrument 104 by a user. An input cassette can hold one or more capped tubes comprising specimen. In some examples, each tube comprises a different specimen (e.g., a specimen from a different patient). The input station 562 is capable of receiving various types of cassettes corresponding to various types of tubes. The input station 562 includes a reader or scanner capable of reading machine-readable codes, such as a barcode, a QR code, or a Radio Frequency Identification (RFID) tag, among other similar examples. In some examples, each tube has an associated code to identify the specimen within the tube that is scanned by the reader or scanner.

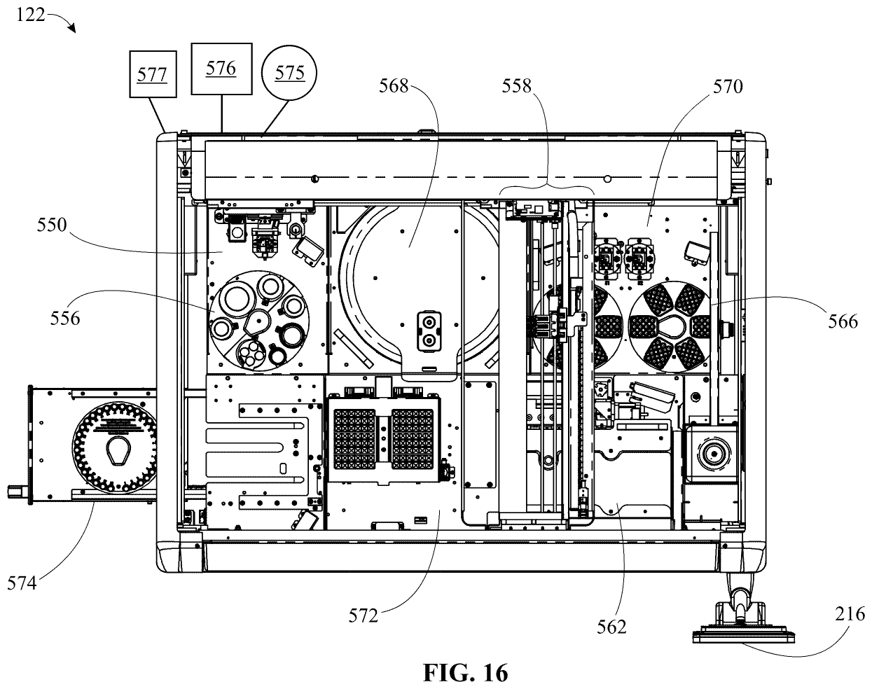

FIG. 16 depicts in a cross-section view examples of the hardware components illustrated and described in FIG. 15. In this illustrated example, in addition to the hardware components described in FIG. 15, FIG. 16 further depicts a waste container 575, a diluent reagent container 576, and a condensation collector 577. Waste associated with the sample preparation process, including the specimen waste from the cell concentration estimator 550 is dispensed to the waste container 575. The diluent reagent can be used in several steps through the preparation process, including the cell concentration estimation and to reconstitute dry reagent, among other examples. The condensation collector 577 can collect any condensation resulting from a cooling of the labeling reagents (e.g., generated by the liquid reagent housing 568).

FIG. 17 is a schematic block diagram illustrating an example of the sample preparation instrument 104. In this illustrated example, the sample preparation instrument 104 includes an example of the sample preparation hardware 122, which includes the sample preparation computer 118, sensors 602, and actuators 604. In some embodiments, the sample preparation computer 118 includes the sample preparation software 124, a sample preparation file storage 582, and sample preparation validation criteria 584. The sample preparation software 124 is executed by and operates on the sample preparation computer 118. Also shown in this illustrated example is the sample preparation specification 116. In some examples, the sample preparation software 124 includes a sample preparation validator 586, sample preparation 588, and a serpentine pattern generator 590. The example sample preparation validator 586 includes a load validator 592, a run validator 594 and a predictive validator 596.

In some embodiments, the sample preparation computer 118 is operable to import the sample preparation specification 116, which is generated and output by the panel designer software 114 illustrated and described in FIG. 4. Via the sample preparation file storage 582, the sample preparation software 124 is operable to take the sample preparation specification 116 as its first input. The sample preparation software 124 is also operable to take the user's selection (for instance, by manual input) as its second input. With inputs including at least the sample preparation specification 116 and the user's selection, the sample preparation software 124 can then generate the sample preparation 588, which can be an ordered list of operations for the sample preparation instrument 104 to carry out.

In some examples, the sample preparation validator 586 can validate the sample preparation 588 against the sample preparation validation criteria 584, which can be provided by the sample preparation file storage 582. The sensors 602 operate to provide information when required by the sample preparation validator 586 for validating. When the sample preparation validator 586 determines that the sample preparation 588 fails to comply with the sample preparation validation criteria 584, the sample preparation validator 586 can generate notifications to alert the user.

In other embodiments, the sample preparation validator 586 can send commands to the sample preparation hardware 122 to change the operation of the sample preparation instrument 104. With the commands, the sample preparation validator 586 is operable to disable, with the help of the actuators 604, the sample preparation 588 from being executed or carried out. For instance, the sample preparation validator 586, using the load validator 592, can cause to prevent the sample preparation instrument 104 from loading the sample to be prepared. In other examples, the sample preparation validator 586, using the run validator 594, can cause the sample preparation instrument 104 to cease operating according to the sample preparation 588. In further examples, the sample preparation validator 586, using the predictive validator 596, can cause the sample preparation instrument 104 to cease operating or loading in consideration of other operations being run or having been scheduled to run on the sample preparation instrument 104.

Examples of the sensors 602 include floats in the waste container 575, the diluent reagent container 576, and the condensation collector 577, optical camera for reagents identification, or any other types of sensors. Examples of the actuators 604 include the input station 562, the probes 560, the single tube loader 564, or any other types of actuators.

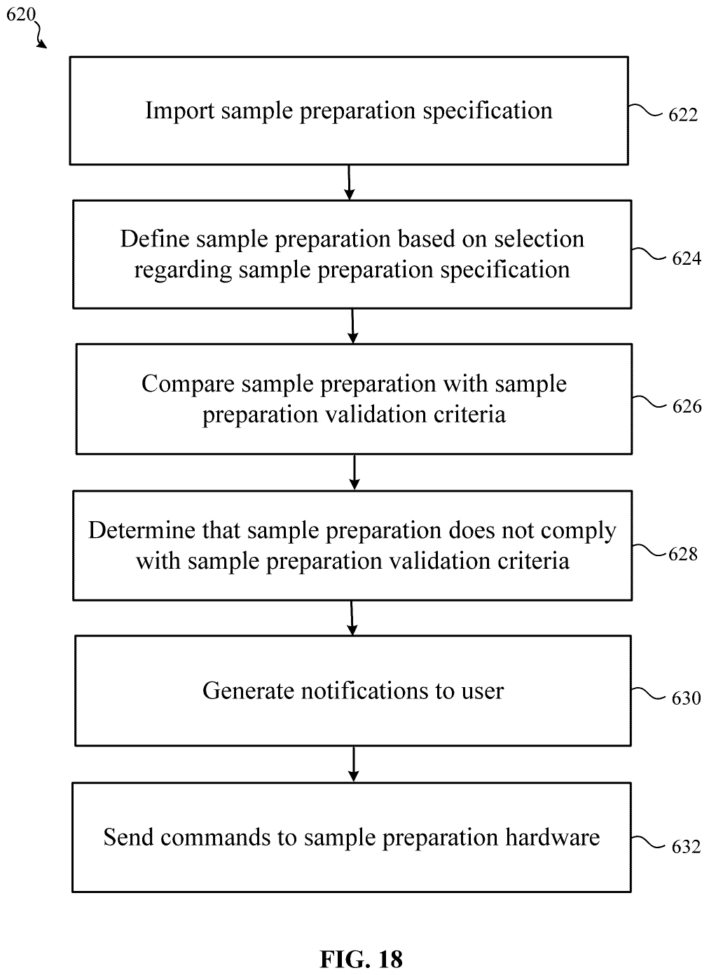

FIG. 18 is a flow chart illustrating an example method 620 of validating sample preparation. In one example, the method 620 is operated by the sample preparation instrument 104, shown in FIG. 17. In this example, the method 620 includes operations 622, 624, 626, 628, 630, and 632.

The operation 622 is performed to import the sample preparation specification 116 for preparing the sample. In some embodiments the operation 622 is performed by the sample preparation computer 118, such as shown in FIG. 17.

The operation 624 is performed to define the sample preparation 588 based on selection that is made regarding the sample preparation specification 116. In some embodiments the operation 624 is performed by the sample preparation software 124, such as shown in FIG. 17.

The operation 626 is performed to compare the sample preparation 588 with the sample preparation validation criteria 584. In some embodiments the operation 626 is performed by the sample preparation validator 586, such as shown in FIG. 17.

The operation 628 is performed to determine whether the sample preparation 588 does not comply with the sample preparation validation criteria 584. In some embodiments the operation 628 is performed by the sample preparation validator 586, such as shown in FIG. 17.

The operation 630 is performed to generate notifications to the user according to the determination made by the operation 628. In some embodiments the operation 630 is performed by the sample preparation validator 586, such as shown in FIG. 17.

The operation 632 is performed to send commands to the sample preparation hardware 122 to change the operation of the sample preparation instrument 104, according to the determination made by the operation 628. In some embodiments the operation 632 is performed by the sample preparation validator 586, such as shown in FIG. 17.

FIG. 19 illustrates and describes an example user interface 650 for using the sample preparation validator 586, such as shown in FIG. 17, to check and validate the sample preparation 588. In this illustrated example, the example user interface 650 includes a sample preparation ID field 652 and a sample preparation error status indicator 654. If the sample preparation defined by the sample preparation software 124 that corresponds to the data (for instance, an identification string, etc.) in the sample preparation ID field 652 is determined by the sample preparation validator 586 to contain errors, the sample preparation error status indicator 654 operates to notify the user of the error status. For instance, the sample preparation error status indicator 654 can display an icon indicating errors.

In some embodiments, the example user interface 650 displays a sample preparation error window 656 containing information about all errors found by the sample preparation validator 586 from the currently selected sample preparation. The sample preparation error window 656 also corresponds to the currently selected sample preparation ID field 652. In some examples, the user interface 650 includes a sample preparation remove button 658. By clicking on the sample preparation remove button 658, the user can remove a corresponding sample preparation from the current selections. In other examples, the user interface 650 displays a global error status indicator 660. When any selected sample preparation is determined by the sample preparation validator 586 to contain any error, the global error status indicator 660 operates to notify the user of the error status. For instance, the global error status indicator 660 can display an icon indicating errors. In an exemplary scenario, the user can use the sample preparation remove button 658 to remove all sample preparation selections having errors. When all sample preparation remaining in selection is error-free, the global error status indicator 660 can change its indication to reflect the status change.

In other embodiments, the example user interface 650 includes a load button 662. The user can click the load button 662 to send commands to the sample preparation instrument 104 to execute on loading a sample to be prepared. When the sample preparation validator 586 determines that the currently selected sample preparation contains any error, the sample preparation validator 586 can disable the load button 662 preventing the user from sending commands to load. In an exemplary scenario, when the global error status indicator 660 indicates an error status, the load button 662 will be disabled.

In some examples, the validations to be checked by the sample preparation validator 586 using the load validator 592 include checking the error status variable of the sample preparation specification 116, checking the file format and hash code of the sample preparation specification 116, checking white blood cells concentration in the sample against the user-input WBC range of the sample preparation specification 116, checking presence and expiration date of the sample preparation consumables 92, checking the status of the sample preparation hardware 122 (for instance, the instrument syringe QC state), checking the reagent type of any custom reagent, and checking the sample preparation specification 116 for the order of tubes, blocks, and reagents against expected index values. In further examples, additional rules checked by the load validator 592 include that all blocks of the sample preparation specification 116 must contain at least one reagent or a single reagent kit.

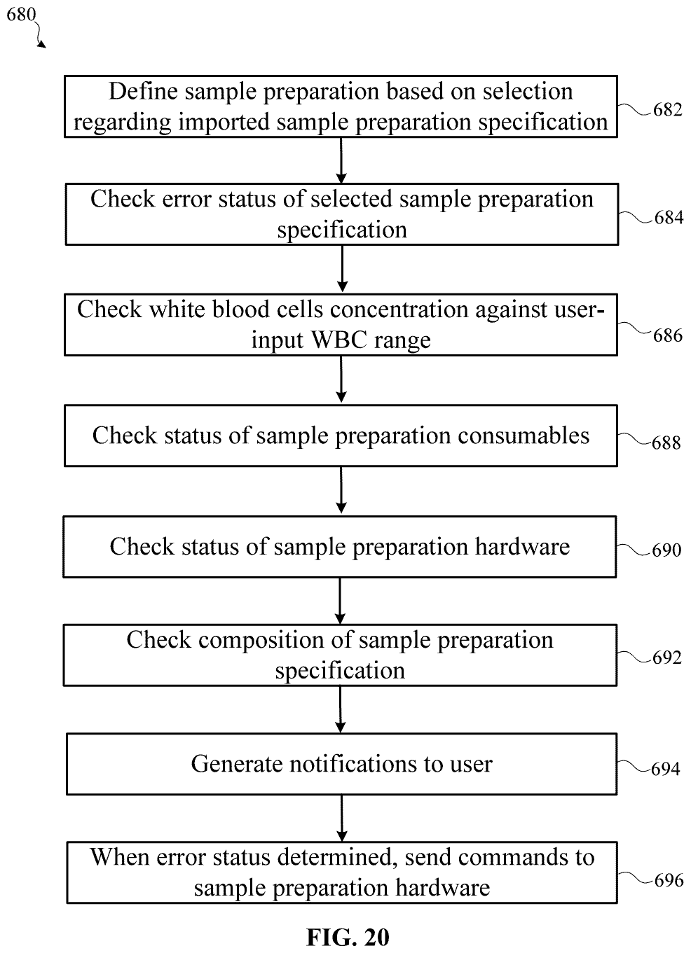

FIG. 20 is a flow chart illustrating an example method 680 of validating at least a portion of the sample preparation 588 at the point of loading specimen. In one example, the method 680 is operated by the load validator 592, shown in FIG. 17. In this example, the method 680 includes operations 682, 684, 686, 688, 690, 692, 694, and 696.

The operation 682 is performed to define the relevant portion of the sample preparation 588 based on selection regarding the imported sample preparation specification 116.

In some embodiments, any combination of one or more of the operations 684, 686, 688, 690, and 692 can be performed. The operation 684 can be performed to check error status of the selected sample preparation specification 116. The operation 686 can be performed to check white blood cells concentration against user-input WBC range contained in the sample preparation specification 116. The operation 688 can be performed to check status of the sample preparation consumables 92. For instance, the load validator 592 is operable to check the volume of the remaining supply in the diluent reagent container 576 by using information acquired by a float sensor. The operation 690 can be performed to check status of the sample preparation hardware 122. The operation 692 can be performed to check the composition of sample preparation specification 116. For instance, the load validator 592 is operable to check whether the order of tubes, reagent blocks, and reagents complies with expected index values.

In other embodiments, the operation 694 is performed to generate notifications to alert the user of any error status. When error status is determined by the load validator 592, the operation 696 is performed to send commands to the sample preparation hardware 122 and cause the sample preparation hardware 122 to operate according to the errors found.

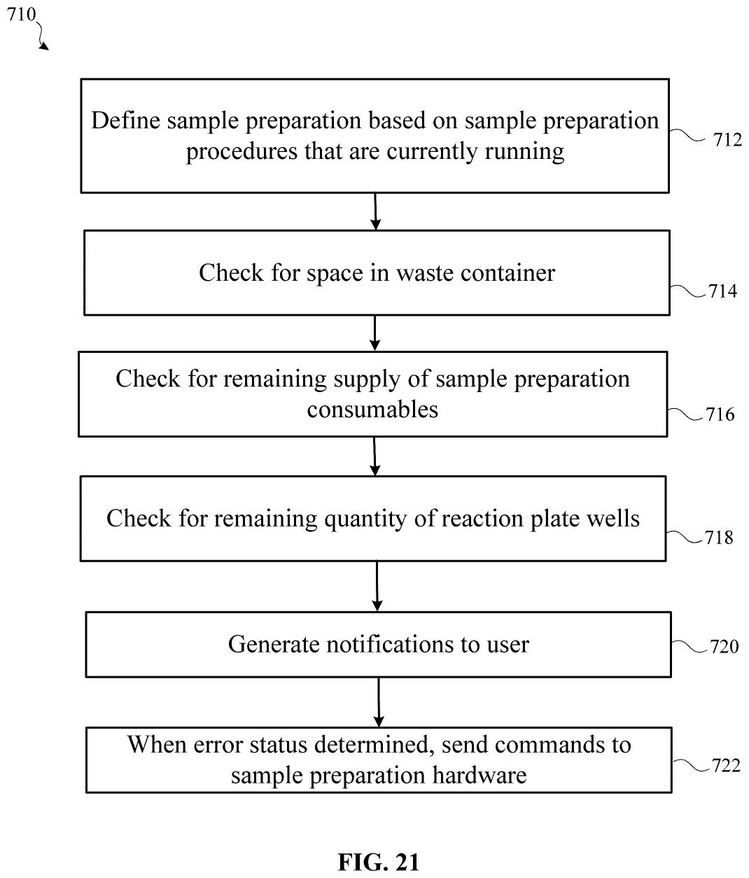

FIG. 21 is a flow chart illustrating an example method 710 of validating at least a portion of the sample preparation 588 when the sample preparation instrument 104 is running procedures to prepare one or more samples. In one example, the method 710 is operated by the run validator 594, shown in FIG. 17. In this example, the method 710 includes operations 712, 714, 716, 718, 720, and 722.

The operation 712 is performed to define the relevant portion of the sample preparation 588 based on the sample preparation procedures that are currently running.

In some embodiments, any combination of one or more of the operations 714, 716, and 718 can be performed. The operation 714 can be performed to check for space in the waste container 575. For instance, the run validator 594 is operable to check the remaining space in the waste container 575 by using information acquired by a float sensor. The operation 716 can be performed to check the remaining supply of the sample preparation consumables 92. For instance, the run validator 594 is operable to check the volume of the remaining supply in the diluent reagent container 576 by using information acquired by a float sensor. The operation 718 can be performed to check the remaining quantity of available reaction plate wells in the reaction station 554.

In other embodiments, the operation 720 is performed to generate notifications to alert the user of any error status. When error status is determined by the run validator 594, the operation 722 is performed to send commands to the sample preparation hardware 122 and cause the sample preparation hardware 122 to operate according to the errors found.

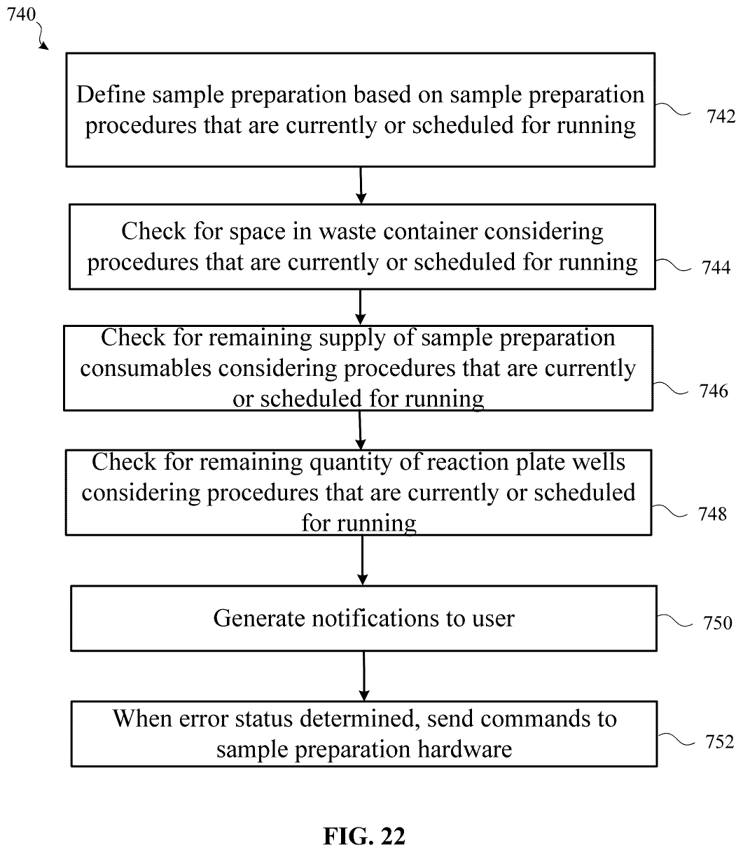

FIG. 22 is a flow chart illustrating an example method 740 of predictively validating at least a portion of the sample preparation 588. In one example, the method 740 is operated by the predictive validator 596, shown in FIG. 17. In this example, the method 740 includes operations 742, 744, 746, 748, 750, and 752.

The operation 742 is performed to define the relevant portion of the sample preparation 588 based on the sample preparation procedures that are currently running or scheduled for running.

In some embodiments, any combination of one or more of the operations 744, 746, and 748 can be performed. The operation 744 can be performed to predictively check for space in the waste container 575 considering procedures that are currently or scheduled for running. For instance, the predictive validator 596 is operable to predict, using computer-executable algorithms and information acquired by a float sensor in the waste container 575, the remaining space after the currently running and scheduled procedures are completed.

The operation 746 can be performed to predictively check the remaining supply of the sample preparation consumables 92. For instance, the predictive validator 596 is operable to predict, using computer-executable algorithms and information acquired by a float sensor in the diluent reagent container 576, the volume of the remaining supply after the currently running and scheduled procedures are completed.

The operation 748 can be performed to predictively check, using computer-executable algorithms and information acquired by a sensor, the remaining quantity of available reaction plate wells in the reaction station 554 after the currently running and scheduled procedures are completed.

In other embodiments, the operation 750 is performed to generate notifications to alert the user of any error status. When error status is determined by the predictive validator 596, the operation 752 is performed to send commands to the sample preparation hardware 122 and cause the sample preparation hardware 122 to operate according to the errors found.

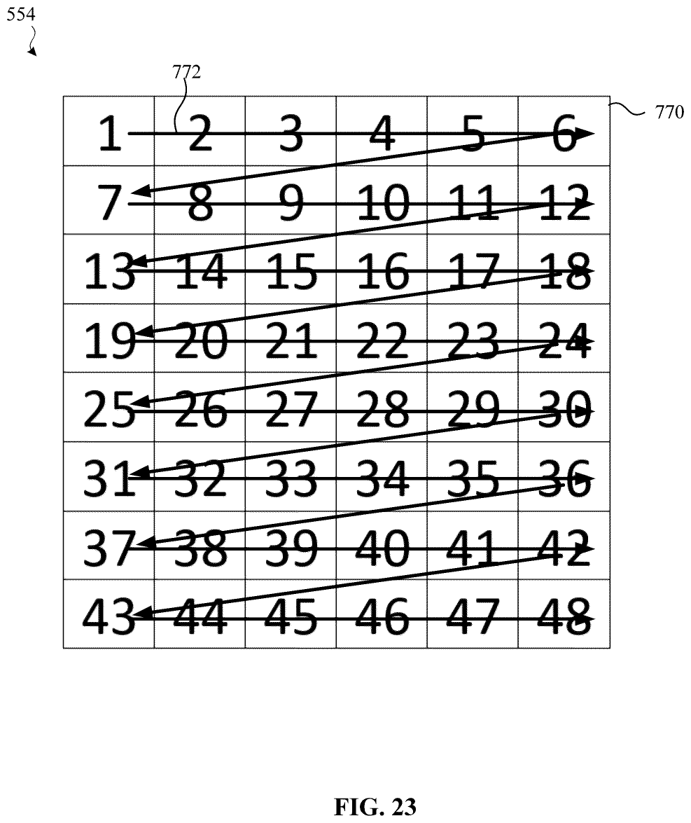

FIG. 23 is a diagram illustrating an example configuration of the probes 560 in the transfer station 558 for using the reaction plate wells in the reaction station 554. In some embodiments, the sample preparation instrument 104 operates to transfer samples to reaction plate wells in the reaction station 554 from, for example, the input station 562 or the cell washer 566.

In other embodiments, when a sample is to be transferred to more than one well, the sample preparation instrument 104 operates to aspirate the sum of all the volumes required for each well on one aspiration. Then, the sample preparation instrument 104 operates to dispense the individual volumes in the wells. For example, when 100 microliters of a sample are needed in each of four wells in the reaction station 554, the probes 560 will aspirate 400 microliters plus some additional conditioning volume. The probes 560 operate to throw away a small amount of the conditioning volume at the probe wash station 573. Then, the probes 560 operate to move to the reaction station 554 and add 100 microliters to the first well, 100 microliters to the second well, 100 microliters to the third well, and 100 microliters to the fourth well.

In the example illustrated in FIG. 23, the reaction station 554 includes forty-eight wells 770 numbered from 1 through 48 as illustrated. When transferring samples, the sample preparation computer 118 operates to control the probes 560 to move in a pattern as illustrated by the arrows 772. For instance, the probes 560 are configured to sequentially transfer samples to fill wells 770 from number 1 through number 48.

When transferring a sample to multiple wells according to a pattern illustrated by FIG. 23. A small drop at the tip of one of the probes 560 may fall while the probe was traveling over another well that contains a different sample or is planned to contain a different sample. As a result, the drop would contaminate that different sample. Therefore, a different pattern of moving the probes 560 is desirable to reduce cross-contamination among different samples in the wells of the reaction station 554.

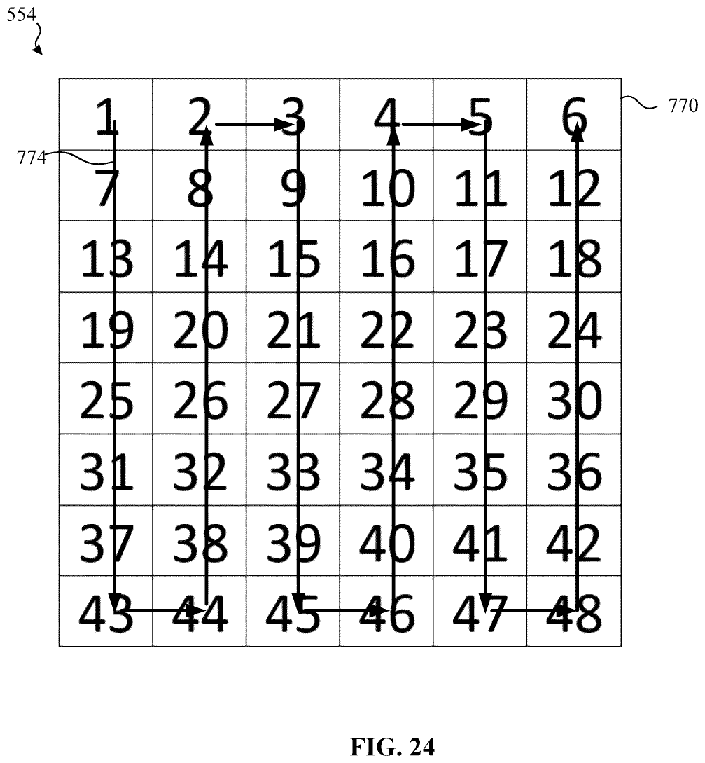

FIG. 24 is a diagram illustrating another example configuration of the probes 560 in the transfer station 558 for using the reaction plate wells in the reaction station 554. In this illustrated example, the sample preparation computer 118 operates to control the probes 560 to move in a pattern as illustrated by the arrows 774. For instance, the probes 560 are configured to transfer samples to fill wells 770 in the order of number 1, 7, 13, 19, 25, 31, 37, 43, 44, 38, 32 . . . 4, 5, 11, 17, 23, 29, 35, 41, 47, 48, 42, 36, 30, 24, 18, 12, and 6. The serpentine pattern as illustrated by the arrows 774 is operable to cause the probes 560 to travel over wells that contain the same sample. As a result, cross-contamination from one sample to another is mitigated. The serpentine pattern is not only implemented for sample transfers. All specimens, samples, and reagents are configured to be transferred to the wells in the reaction station 554 in this serpentine pattern. By not treating sample transfers as a special case, software complexity of the sample preparation software 124 can be reduced.

In some embodiments, the serpentine pattern generator 590 operates to generate the serpentine pattern illustrated in FIG. 24.

In other embodiments, after transferring samples, the probes 560 are configured to travel to the probe wash station 573 described in FIG. 15 to be washed.

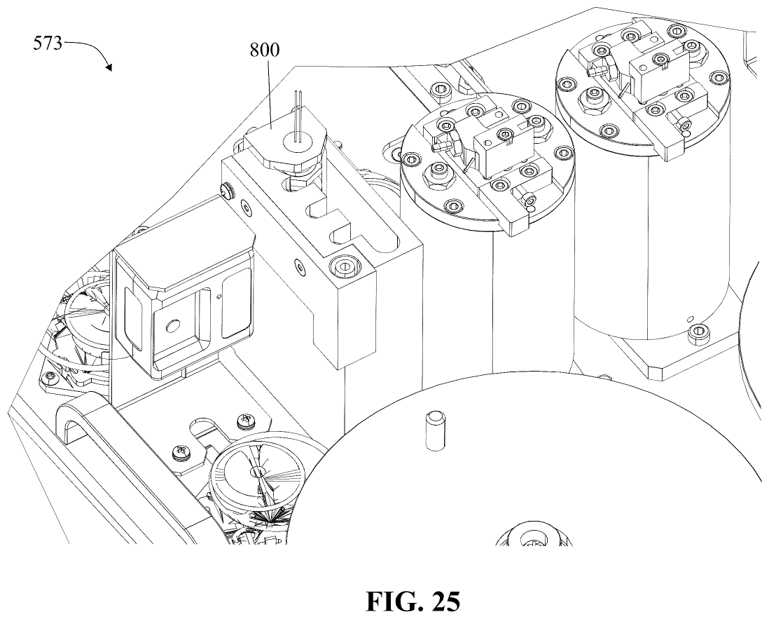

FIG. 25 is a diagram depicting a portion of the sample preparation hardware 122 near the probe wash station 573, the probe wash station 573 containing a probe wash module 800.

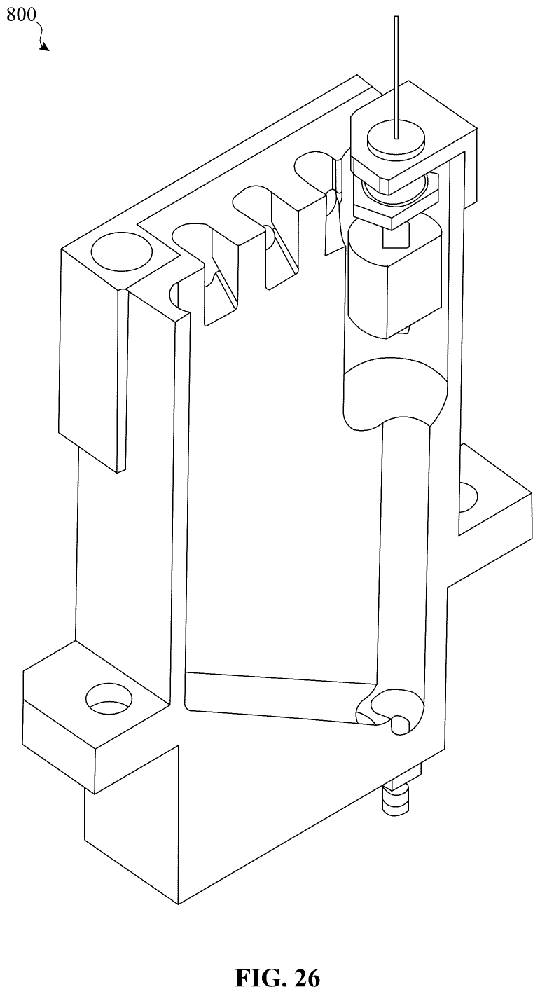

FIG. 26 is a close-up view depicting the probe wash module 800. The probe wash module 800 includes a trough for receiving unused sample or reagent fluid dispensed from a number of probes, as well as sheath or washed fluid dispensed from the probes in order to clean the inside surfaces of the probe. The fluid in this trough is drawn from an exit port at the bottom of the trough by a pump and delivered to a bulk waste container outside the instrument.

In some embodiments, the probe wash module 800 also includes one or more wells adjacent to the trough for washing the outside surface of the probe. The probe is lowered into the one or more wells and filled with sheath or wash solution from the probe. This dispensed sheath or wash fluid overflows into the trough and is then pumped out to the bulk waste container. The flow of the fluid in the well during its dispense assists the washing of the outside surface. In this illustrated example, the probe wash module 800 includes three wells for washing a three-channel probe.

In other embodiments, the trough of the probe wash module 800 includes an internal chamber with a level-sensing device, such as a float valve, serving as a safety shut-off valve to prevent the dispensed sheath or wash fluid from overflowing onto the deck of the instrument if there is a blockage of flow between the probe wash module 800 and the bulk waste reservoir.

This disclosure should be understood to include (as illustrative and not limiting) the subject matter set forth in the following numbered clauses:

Clause 1. A panel design system comprising a computing device and panel designer software, the panel designer software being executable by the computer to cause the panel design system to:
generate a sample preparation specification; and
validate the sample preparation specification.

Clause 2. The panel design system of claim 1, the panel designer software being further executable to output the sample preparation specification to a file.

Clause 3. A panel validation method comprising:
defining a user-input specification;
comparing the user-input specification with validation criteria;
determining that the user-input specification does not comply with the validation criteria;
generating at least one notification; and
generating and outputting a sample preparation specification.

Clause 4. The panel validation method of claim 3, wherein the user-input specification includes a white blood cell range specification and a reagents specification.

Clause 5. The panel validation method according to any one of claims 3-4, further comprising any combination of one or more of the following:
checking a first data about at least one WBC concentration upper limit; or
checking a second data about at least one specimen volume.

Clause 6. The panel validation method according to claim 5, further comprising any combination of one or more of the following:
highlighting at least a portion of the first data and the second data, the portion of the first data and the second data being determined to contain at least an error;
displaying information about the error; or
listing the error in a message.

Clause 7. The panel validation method according to any one of claims 3-6, further comprising any combination of one or more of the following:
checking a first data about at least one reagent name;
checking a second data about at least one reagent volume;
checking a third data about at least one incubation duration; or
checking a fourth data about at least one resuspend option.

Clause 8. The panel validation method of claim 7, further comprising any combination of one or more of the following:
highlighting at least a portion of the first data, the second data, the third data, and the fourth data, the portion of the data being determined to contain at least an error;
displaying information about the error; or
listing the error in a message.

Clause 9. A sample preparation instrument comprising sample preparation hardware and sample preparation software, the sample preparation software being executable by a computer of the sample preparation hardware to cause the sample preparation instrument to:
receive a sample preparation specification;
define sample preparation based on at least the sample preparation specification;
validate sample preparation; and
generate a prepared sample based on the sample preparation.

Clause 10. The sample preparation instrument of claim 9, the sample preparation software being further executable to cause the sample preparation instrument to:
validate an error status of the sample preparation specification.

Clause 11. A sample preparation method comprising:
receiving a sample preparation specification;
defining a sample preparation based on at least the sample preparation specification;
comparing the sample preparation with sample preparation validation criteria;
determining that the sample preparation does not comply with the sample preparation validation criteria;
generating at least one notification; and sending at least one command to sample preparation hardware.

Clause 12. The sample preparation method of clause 11, further comprising any combination of one or more of the following:
checking an error status of the sample preparation specification;
checking a first data about at least one white blood cells concentration;
checking a first status of at least one sample preparation consumable; or
checking a second status of sample preparation hardware.

Clause 13. The sample preparation method of any one of clauses 11 or 12, further comprising any combination of one or more of the following:
generating at least one notification; or
sending at least one command to the sample preparation hardware.

Clause 14. The sample preparation method of any of claims 11-13, wherein the sample preparation specification is received by a sample preparation instrument via a data transfer system.

Clause 15. The sample preparation method of any one of clauses 11-14, further comprising, preparing a sample using sample preparation consumables.

Clause 16. The sample preparation method of any one of clauses 11-15, wherein the sample preparation specification is defined by input from an operator.

Clause 17. The sample preparation method of clause 12, wherein the error status is indicated by a flag variable having a value that may be “true” or “false.”

Clause 18. The sample preparation method of any one of clauses 11-17, wherein the sample preparation specification contains a hash code.

Clause 19. The sample preparation method of clause 18, wherein the hash code is used to verify that no unauthorized modifications have been made to the sample preparation specification.

Clause 20. The sample preparation method of clause 14, wherein the sending at least one command to the sample preparation hardware causes the sample preparation hardware to change the operation of the sample preparation instrument.

Clause 21. The sample preparation method of clause 20, wherein the sending at least one command to the sample preparation hardware causes the sample preparation hardware to disable sample preparation from being executed.

Clause 22. The sample preparation method of any one of clauses 11-21, further comprising displaying an icon indicating errors on a sample preparation error status indicator.

Clause 23. The sample preparation method of any one of clauses 11-22, further comprising displaying a sample preparation error window containing information about errors in a sample preparation.

Clause 24. The sample preparation method of any one of clauses 11-23, wherein the comparing the sample preparation with the sample preparation validation criteria comprises checking white blood cells concentration in the sample preparation against the user-input WBC range of the sample preparation specification.

Clause 25. The sample preparation method of clause 15, wherein the comparing the sample preparation with the sample preparation validation criteria comprises checking presence and expiration date of the sample preparation consumables.

Clause 26. A sample analysis system comprising:
a panel design system that generates a sample preparation specification;
a sample preparation instrument that prepares a sample using the sample preparation specification to generate a prepared sample;
a sample analyzer configured to analyze the prepared sample; and
at least one validator configured to validate an operation of the sample analysis system.

Clause 27. A sample analysis system comprising:
the panel design system of clause 1; and
the sample preparation instrument of clause 9.

Clause 28. The sample analysis system of clause 27, further comprising:
a sample analyzer configured to analyze the prepared sample generated by the sample preparation instrument.

Clause 29. The sample analysis system of clause 28, wherein the sample analyzer is a flow cytometer.

Clause 30. The sample analysis system of any one of clauses 28-29, wherein the sample analyzer is a hematology analyzer.

Clause 31. A method comprising:
the panel validation method of clause 3; and
the sample preparation method of clause 11.

Clause 32. A non-transitory computer readable storage media of a sample analysis system, the computer readable storage media storing data instructions that, when executed by a processing device, cause the sample analysis system to perform operations according to any one of clauses 3-8, 11-25, or 31.

Clause 33. A panel design system that compares a user-input specification with validation criteria, and generates a notification when the user-input specification does not comply with the validation criteria.

Clause 34. The panel design system of clause 33, wherein the notification is selected from: highlighting a field of the user-input specification with a color-coded indicator; displaying an information box based on a pointer located on the corresponding data field; and displaying an error message.

Clause 35. A sample preparation instrument that evaluates sample preparation using a sample preparation specification and generates a notification upon detecting an error.

Clause 36. The sample preparation instrument of clause 35, wherein detecting the error is detected based on a predicted operation of the sample preparation instrument using the sample preparation specification and a current status of the sample preparation instrument.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

1. A sample preparation method comprising:
receiving a sample preparation specification;
defining a sample preparation based on at least the sample preparation specification;
comparing the sample preparation with sample preparation validation criteria;
determining that the sample preparation does not comply with the sample preparation validation criteria;
generating at least one notification; and
sending at least one command to sample preparation hardware.

2. The sample preparation method of claim 1, further comprising any combination of one or more of the following:
checking an error status of the sample preparation specification;
checking a first data about at least one white blood cells concentration;
checking a first status of at least one sample preparation consumable; or
checking a second status of sample preparation hardware.

3. The sample preparation method of claim 1, further comprising any combination of one or more of the following:
generating at least one notification; or
sending at least one command to the sample preparation hardware.

4. The sample preparation method of claim 1, wherein the sample preparation specification is received by a sample preparation instrument via a data transfer system.

5. The sample preparation method of claim 1, further comprising, preparing a sample using sample preparation consumables.

6. The sample preparation method of claim 1, wherein the sample preparation specification is defined by input from an operator.

7. The sample preparation method of claim 2, wherein the error status is indicated by a flag variable having a value that may be “true” or “false.”

8. The sample preparation method of claim 1, wherein the sample preparation specification contains a hash code.

9. The sample preparation method of claim 8, wherein the hash code is used to verify that no unauthorized modifications have been made to the sample preparation specification.

10. The sample preparation method of claim 4, wherein the sending at least one command to the sample preparation hardware causes the sample preparation hardware to change the operation of the sample preparation instrument.

11. The sample preparation method of claim 10, wherein the sending at least one command to the sample preparation hardware causes the sample preparation hardware to disable sample preparation from being executed.

12. The sample preparation method of claim 1, further comprising displaying an icon indicating errors on a sample preparation error status indicator.

13. The sample preparation method of claim 1, further comprising displaying a sample preparation error window containing information about errors in a sample preparation.

14. The sample preparation method of claim 1, wherein the comparing the sample preparation with the sample preparation validation criteria comprises checking white blood cells concentration in the sample preparation against the user-input WBC range of the sample preparation specification.

15. The sample preparation method of claim 5, wherein the comparing the sample preparation with the sample preparation validation criteria comprises checking presence and expiration date of the sample preparation consumables.
